(19) United States
(12) Patent Application Publication
(10) Pub. No.: US 2025/0009973 A1
(43) Pub. Date: Jan. 9, 2025

(54) INJECTION APPARATUS AND METHOD

(71) Applicant: Bruce Dixon, Orlando, FL (US)

(72) Inventor: Bruce Dixon, Orlando, FL (US)

(21) Appl. No.: 18/764,476

(22) Filed: Jul. 5, 2024

Related U.S. Application Data
(60) Provisional application No. 63/525,498, filed on Jul. 7, 2023.

(51) Int. Cl. A61M 5/28; A61M 5/20; A61M 5/315

(52) U.S. Cl. CPC A61M 5/281; A61M 5/31528; A61M 2005/2073

(57) ABSTRACT

Methods and devices useful for intraarticular or other targeted injection of medication, for example, into an anatomical area. A surgical apparatus may facilitate such injections and may also function as a Steinmann pin or an intramedullary pin. The apparatus may have an outer shell and an inner pin. The inner pin and the outer shell may cooperatively engage to provide a first configuration and a second configuration. In the first configuration the inner pin and the outer shell may be aligned along a longitudinal axis, the inner pin may be disposed within the outer shell, allowing other surgical instruments to slide over the apparatus. In the second configuration the inner pin may be disposed outside of the outer shell and a substance may be injected into the outer shell, for example, via a syringe.

### CROSS-REFERENCE TO RELATED APPLICATIONS

This application claims the benefit of Provisional Application No. 63/525,498, filed Jul. 7, 2023, the entire contents of which is hereby incorporated by reference as if fully set forth herein, under 35 U.S.C. § 119(e).

### BACKGROUND

The sacroiliac joints (SI joints) link the sacrum to the ilium. These two joints are made up of the bony structure above the coccyx, known as the sacrum, and the top part of the pelvis, known as the ilium. The sacroiliac joints support the weight of the upper body when standing. Potential causes of sacroiliac pain include arthritis, traumatic injury, pregnancy and post-partum, systemic inflammatory conditions, and infection. Other potential contributors include spinal scoliosis, leg length discrepancy, and previous lumbar spine fusion. Sometimes, there is no clear cause for sacroiliac pain. Sacroiliac joint pain is most commonly felt in the low back and buttock but can also be referred into the thigh and leg.

Minimally invasive procedures exist to implant bone allografts into the SI joint to stabilize and fuse the dysfunctional joint. The entire procedure is often done through a single, small incision on the patient's back. A Steinmann pin or an intramedullary pin may be inserted through the small incision into the SI joint. The pin may then be used as a guide along which other surgical instruments may slide to ensure accurate alignment. Although the pin is well suited for these purposes, administration of targeted medication during the procedure remains difficult. The target areas at which medication would ideally be administered may often be obscured or blocked by the surgical equipment. A need, therefore, exists for an apparatus and a method for intraarticular or other targeted injection of medication.

The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide methods and devices useful for intraarticular or other targeted injection of medication by providing a surgical apparatus for insertion into an anatomical area, such as a joint (e.g. a sacroiliac joint). However, the device is not limited to being used with a sacroiliac joint and can be applied to many other joints as well as non-joint anatomical areas. The device can be used instead of a traditional Steinmann pin.

The apparatus may comprise an outer shell and an inner pin. The outer shell may comprise a threaded end, a tapered end having a first tapered surface, and a smooth segment extending therebetween. The outer shell may define an interior channel. The interior channel may extend through the threaded end, the smooth segment, and the tapered end along a longitudinal axis of the outer shell. The inner pin may comprise a pointed end having a second tapered surface, a knobbed end, and a shaft. The inner pin and the outer shell may cooperatively engage to provide a first configuration and a second configuration. In the first configuration the inner pin and the outer shell may be aligned along the longitudinal axis, the inner pin may be disposed within the outer shell, the knobbed end of the inner pin may abut the threaded end of the outer shell, the pointed end of the inner pin may extend beyond the tapered end of the outer shell, the first tapered surface and the second tapered surface may align at a common angle relative to the longitudinal axis. In the second configuration the inner pin may be withdrawn outside of the outer shell or may otherwise not be disposed within the outer shell.

Various embodiments relate to a method for injecting a substance into an anatomical area (e.g. a sacroiliac joint) via such a surgical apparatus, the method may comprise inserting the surgical apparatus in the first configuration into the anatomical area, removing the inner pin from the outer shell to put the surgical apparatus into the second configuration, and injecting the substance into outer shell and thereby into the anatomical area. The method may further comprise reinserting the inner pin into the outer shell to put the surgical apparatus into the first configuration. The method may further comprise sliding one or more additional surgical instruments or treatments along the outer shell of the surgical apparatus in the first configuration to guide the placement of the additional surgical instruments or treatments within the anatomical area. The method may further comprise removing the surgical apparatus from the anatomical area to leave the additional surgical instruments or treatments within the anatomical area. The method may further comprise affixing an adapter to the outer shell while the surgical instrument is in the second configuration. These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures, which are not to scale and are for illustrative purposes only.

FIG. 1 is a schematic diagram of a surgical apparatus according to various embodiments in a first configuration.

FIG. 2 is a schematic diagram of a surgical apparatus according to various embodiments in a second configuration.

FIG. 3 is a schematic diagram of a portion of a surgical apparatus according to various embodiments in a first configuration.

FIGS. 4A and 4B are schematic diagrams of an adapter for a surgical apparatus according to various embodiments.

FIGS. 4C through 4E are schematic diagrams of the surgical apparatus in the first configuration received within the adapter according to various embodiments.

FIG. 5A is a schematic diagram of a surgical apparatus, in a first configuration, inserted into a sacroiliac joint.

FIG. 5B is a schematic diagram of a surgical apparatus, in a second configuration, inserted into a sacroiliac joint with an adapter connected thereto to facilitate an injection from a syringe according to various embodiments.

FIG. 5C is a schematic diagram of a surgical apparatus, in a first configuration, inserted into a sacroiliac joint with an additional surgical instrument or treatment sliding over the device.

FIG. 6 is a block diagram illustrating a method for injecting a substance into a sacroiliac joint via a surgical apparatus according to various embodiments.

FIG. 7 is a schematic diagram illustrating a cross-sectional view of a surface having surface irregularities.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

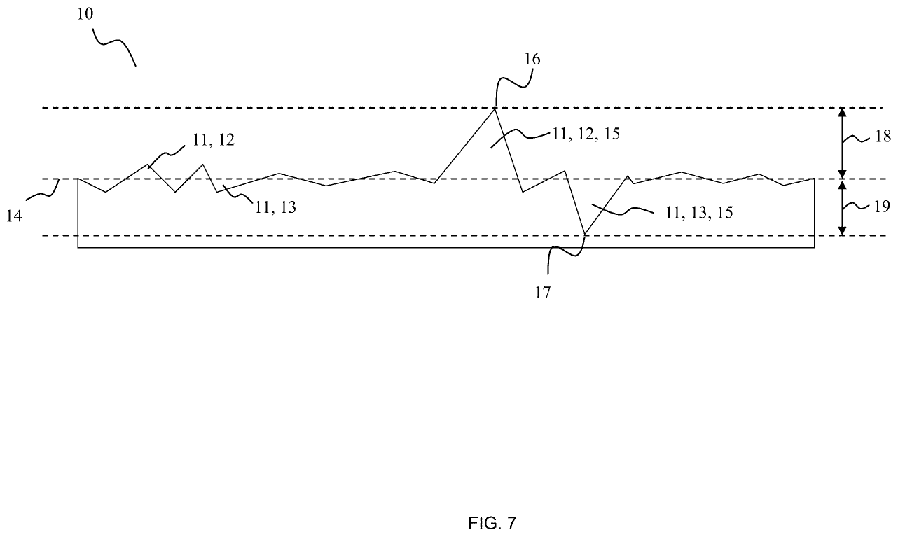

“Surface irregularity” means any deviation in height above or below an average surface level and may include, for example, protrusions, bumps, ridges, grooves, indentions, and combinations thereof. FIG. 7 provides an illustration.

### Overview

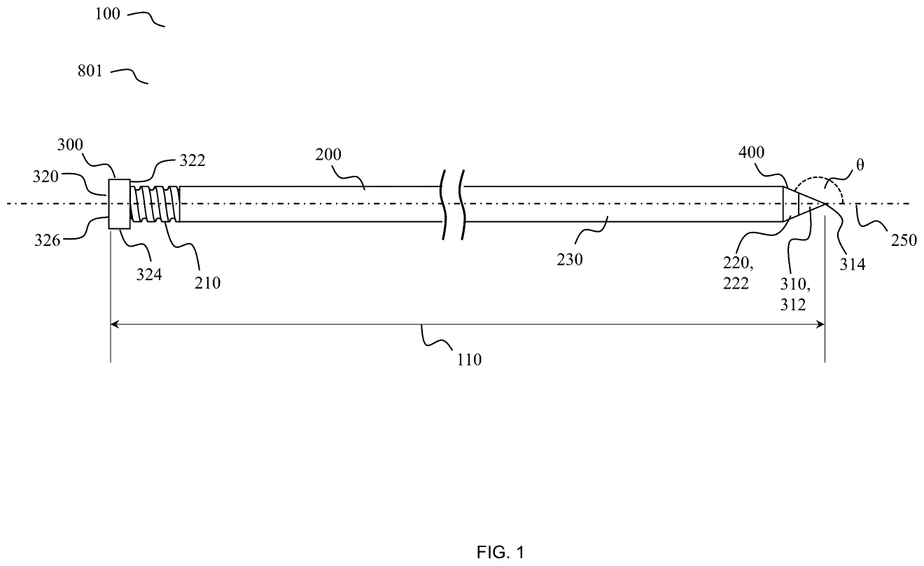

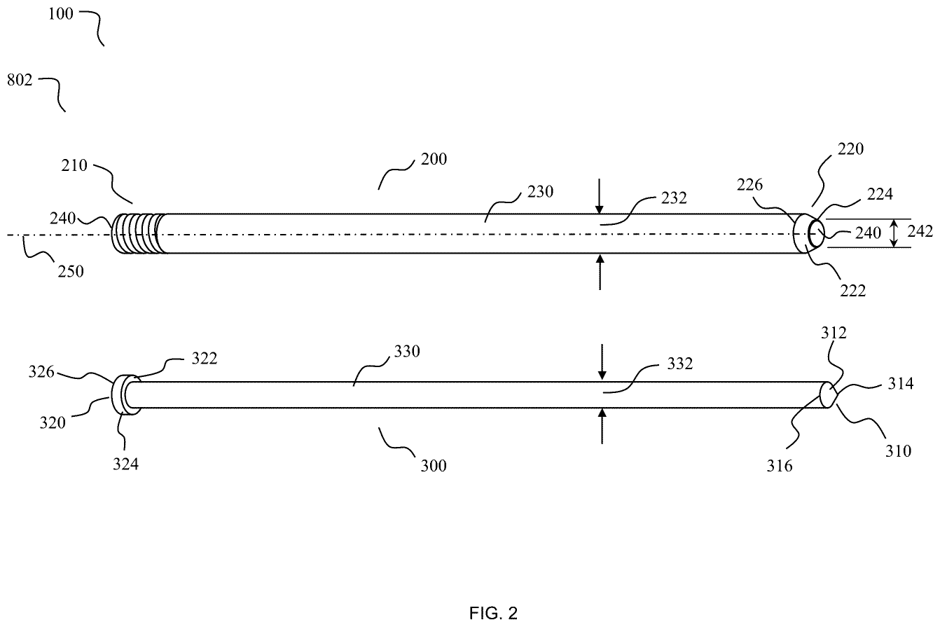

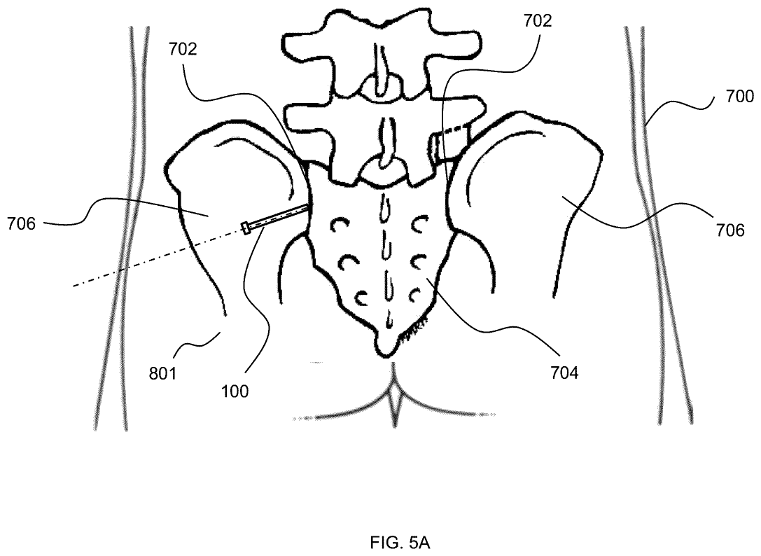

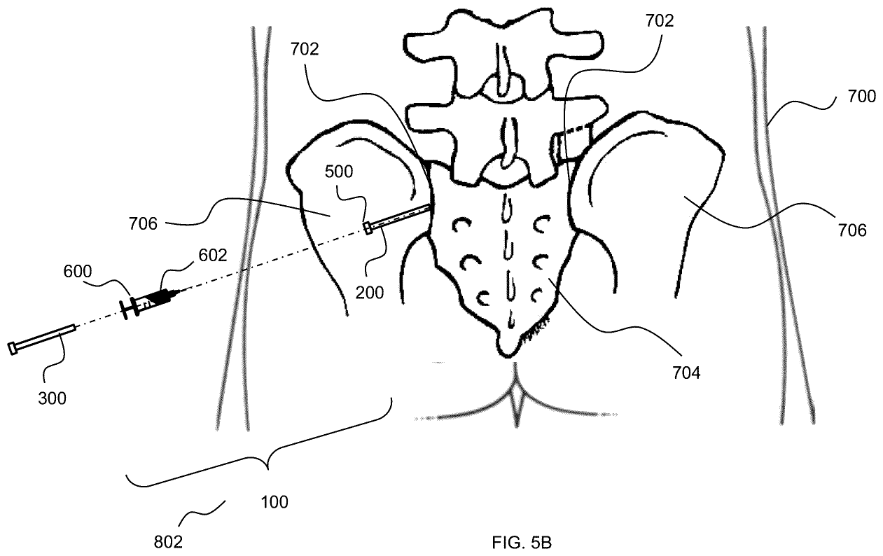

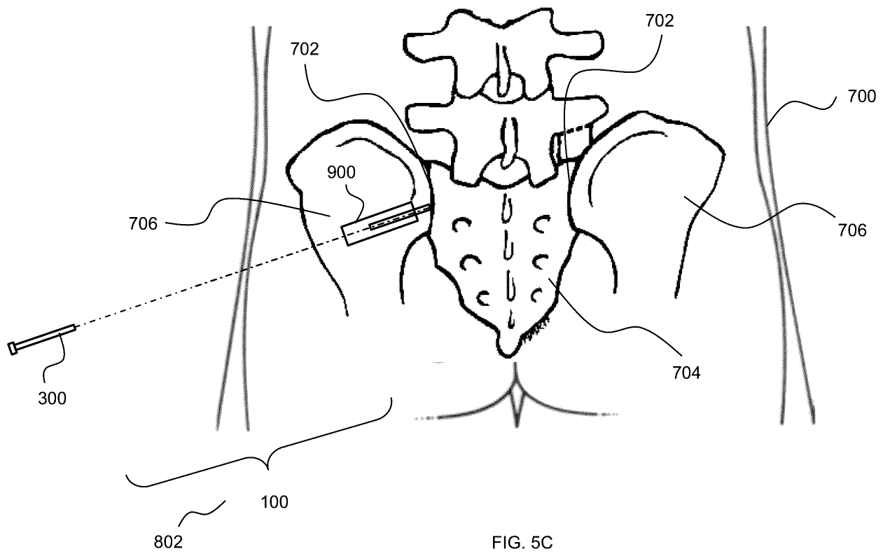

FIG. 1 is a schematic diagram of a surgical apparatus 100, according to various embodiments, for insertion into an anatomical area, such as a joint (e.g. sacroiliac joint 702) (See: FIGS. 5A, 5B, 5C). It is to be appreciated that the apparatus 100 is not shown to scale and that FIG. 1 shows a foreshortened illustration of the apparatus to better illustrate its various features. The apparatus 100 may comprise an outer shell 200 and an inner pin 300. The inner pin 300 and the outer shell 200 may cooperatively engage to provide a first configuration 801 and cooperatively disengage to provide a second configuration 802 (See: FIG. 2). The outer shell 200 may comprise a threaded end 210, a tapered end 220 having a first tapered surface 222, and a smooth segment 230 extending therebetween. The outer shell 200 may define an interior channel 240. The interior channel 240 may extend through the threaded end 210, the smooth segment 230, and the tapered end 220 along a longitudinal axis 250 of the outer shell 200. The inner pin 300 may comprise a pointed end 310 having a second tapered surface 312, a knobbed end 320, and a shaft 330. The knobbed end 320 may provide a grip surface 324 that may optionally be textured to enable a user to grip the knobbed end 320 of the inner pin 300 to insert it or to remove it from the outer shell 200.

In the first configuration 801, as shown in FIG. 1, the inner pin 300 and the outer shell 200 are aligned along the longitudinal axis 250, the inner pin 300 is disposed within the outer shell 200, the knobbed end 320 of the inner pin 300 abuts the threaded end 210 of the outer shell 200, the pointed end 310 of the inner pin 300 extends beyond the tapered end 220 of the outer shell 200, the first tapered surface 222 and the second tapered surface 312 align at a common angle θ relative to the longitudinal axis 250 to form a third tapered surface 400, which may comprise the first tapered surface 222 and the second tapered surface 312. The common angle θ is in a range of from greater than about 90 degrees to less than about 180 degrees, or about 100 degrees to about 170 degrees, or about 110 degrees to about 160 degrees, or about 120 degrees to about 150 degrees, or about 130 degrees to about 140 degrees.

The selection of the value of the common angle θ for the first tapered surface 222 and the second tapered surface 312 is based on various factors. In one embodiment, depending on the application, the surgeon may want a tip that is more pointy (e.g. larger value of the common angle θ) versus one that is more dull (e.g. smaller value of the common angle θ). In one example embodiment, if the surgeon prioritizes the avoidance of damage to any blood vessels or a nerve or other sensitive structure in an area, they may opt for a duller tip (e.g. smaller value of the common angle θ). Whereas if the surgeon prioritizes better penetration of the tissue, they may opt for the sharper tip (e.g. larger value of the common angle θ). Depending on the application, the pointy or dull tip may also offer a different tactile feel, which one may be preferred over the other depending on the situation.

In the first configuration 801, an abutment surface 322 of the knobbed end 320 of the inner pin 300 may abut the threaded end 210 of the outer shell 200 to prevent the inner pin 300 from extending further into the interior channel 240 of the outer shell 200. In the first configuration 801, the apparatus 100 may have a length 110 along the longitudinal axis 250 from a tip 314 of the pointed end 310 of the inner pin 300 to a proximal surface 326 of the knobbed end 320 of the inner pin 300 that is in a range of from about 25 mm to about 500 mm, or about 30 to about 400 mm or about 50 mm to about 300 mm.

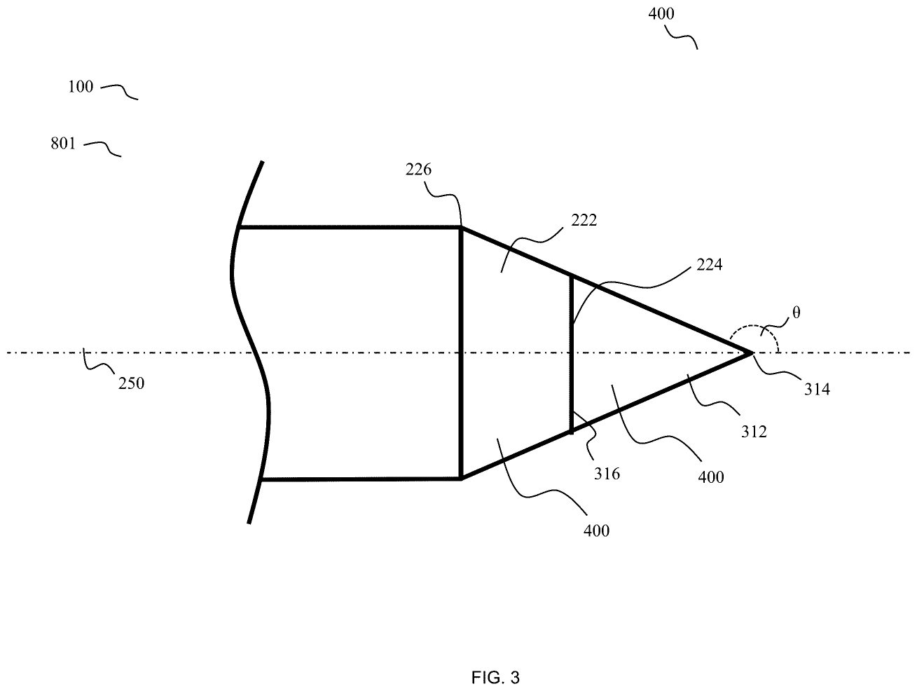

FIG. 2 is a schematic diagram of a surgical apparatus 100 according to various embodiments in the second configuration 802. It is to be appreciated that the apparatus 100 is not shown to scale and that FIG. 2 shows a foreshortened illustration of the apparatus to better illustrate its various features. In the second configuration 802 the inner pin 300 is not disposed within the outer shell 200. The inner pin 300 may have been withdrawn from the outer shell 200 or may have yet to be inserted into the outer shell 200. As can be seen in FIG. 2, the interior channel 240 may be cylindrical. It is to be appreciated that other shapes are possible and are within the scope of this disclosure. The shaft 330 of the inner pin may also be cylindrical. Again, it is to be appreciated that other shapes are possible and are within the scope of this disclosure. An inner diameter 242 of the interior channel 240 may be within about 0.001 mm of an outer diameter 332 of the shaft 330. In other words, a difference between the inner diameter 242 of the interior channel 240 and the outer diameter 332 of the shaft 330 may be in a range of from about 0.0001 mm to about 0.1 mm, or about 0.001 mm to about 0.01 mm. The smooth segment 230 of the outer shell 200 may be cylindrical. The inner diameter 242 may be fixed along a length of the interior channel 240. The outer diameter 332 may be fixed along the length of the shaft 330. The outer diameter 232 of the outer shell 200 may be about 0.01 to about 30 mm, or about 0.1 mm to about 20 mm, or about 0.5 to about 15 mm. According to various embodiments, the smooth segment 230 of the outer shell 200 does not comprise any through-holes, threads, protrusions, or grooves. The inventor recognized that this advantageously gives the smooth segment 230 a lower coefficient of friction. This will allow the smooth segment 230 to pass through tissue optimally with less friction and thus be less destruction of tissue. The inventor recognized that this also allows surgical instruments to glide over the surface without friction. For example, according to various embodiments, the smooth segment 230 of the outer shell 200 may have no surface irregularity 11 having a height or a depth of greater than about 0.0001, or of greater than about 0.001 mm, or of greater than about 0.01 mm. As can be seen in FIG. 2, and as may be better seen in FIG. 3, the outer shell 200 may comprise a first ridge 226 defining a boundary or a transition between the smooth segment 230 and the first tapered surface 222. The outer shell 200 may also comprise an edge 224 defining a boundary or a transition between the first tapered surface 222 and the interior channel 240. Similarly, the inner pin 300 may comprise a second ridge 316 defining a boundary between the shaft 330 and the second tapered surface 312.

FIG. 3 is a schematic diagram of a portion of a surgical apparatus 100 according to various embodiments in the first configuration 801. In the first configuration 801, the first tapered surface 222 and the second tapered surface 312 align at the common angle θ relative to the longitudinal axis 250 to form a third tapered surface 400, and wherein the third tapered surface 400 has no surface irregularity having a height or a depth of greater than about 0.0001, or of greater than about 0.001 mm, or of greater than about 0.01 mm. This means that the second ridge 316 of the inner pin 300 and the edge 224 of the outer shell 200 must be precisely machined to facilitate a smooth transition. In one example, an outer surface of the second ridge 316 is machined so to be oriented at the common angle θ relative to the longitudinal axis 250.

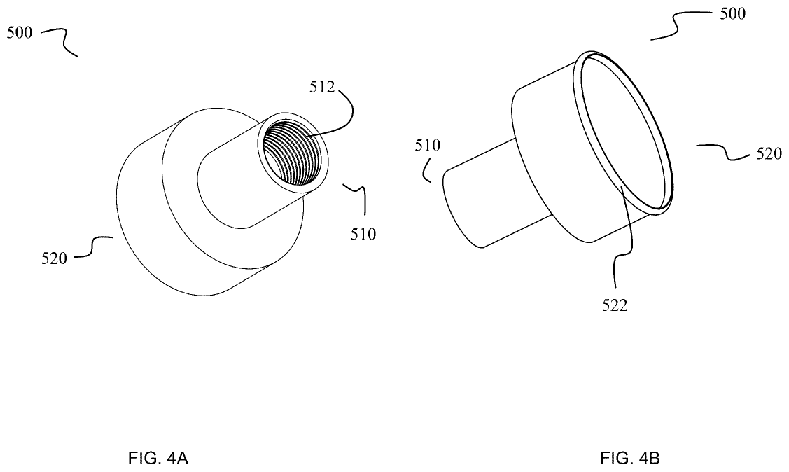

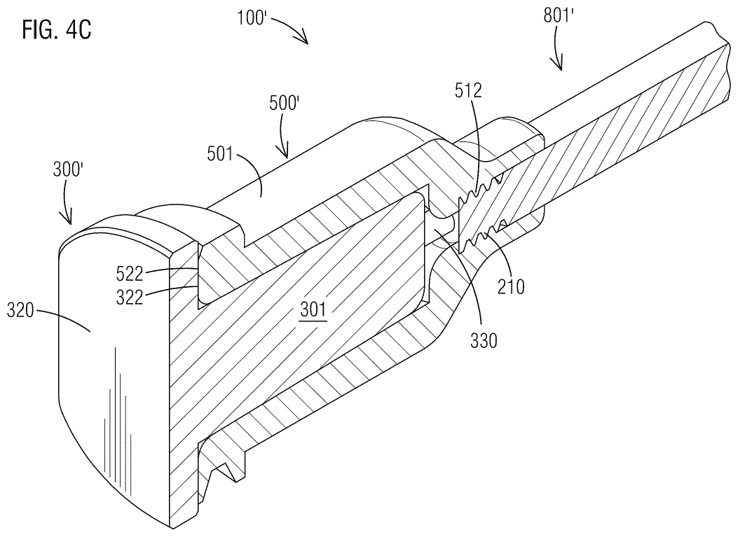

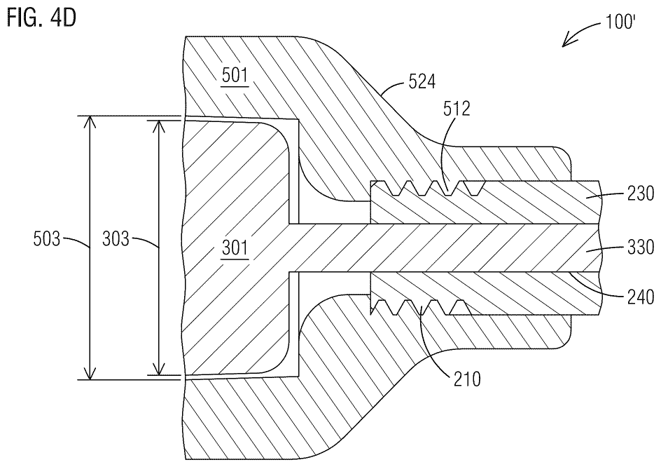

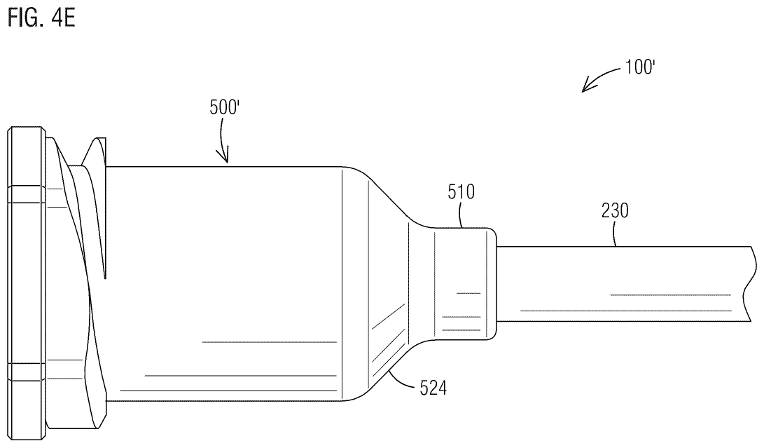

FIGS. 4A and 4B are schematic diagrams of an adapter 500 for the surgical apparatus 100 according to various embodiments. The adapter 500 may comprise a first end 510 and a second end 520. The first end 510 may be configured to engage, for example, to threadedly engage the threaded end 210 of the outer shell 200, via female threads 512. The second end 520 may be configured to engage a syringe 600. Engagement between the second end 520 and the syringe 600 may be accomplished via any suitable means and may be a direct connection or an indirect connection via one or more intermediate adapters. For example, the second end 520 may optionally comprise a lip 522 to engage a syringe 600 or an intravenous drip needle. The lip 522 may, for example, be part of a female Leur lock. It is to be appreciated that a variety of connection may be employed.

FIGS. 4C through 4E are schematic diagrams of an adapter 500′ for a surgical apparatus 100′ according to various embodiments. In one embodiment, the adapter 500′ is attached to the surgical apparatus 100′ in the first configuration 801′. In this embodiment, unlike the first configuration 801 of FIGS. 1 through 3 where the abutment surface 322 of the inner pin 300 abuts the threaded end 210 of the outer shell 200, the first configuration 801′ of FIGS. 4C through 4E features the outer shell 200 and inner pin 300′ aligned along the longitudinal axis 250 but the abutment surface 322 and threaded end 210 are not in direct contact. Instead, the threaded end 210 of the outer shell 200 and abutment surface 322 of the inner pin 300′ are engaged to respective ends 510, 520 of the adapter 500′ while the outer shell 200 and inner pin 300′ remain aligned along the longitudinal axis 250. The adapter 500′ of FIGS. 4C through 4E is similar to the adapter 500 of FIGS. 4A and 4B, with the exception of the features discussed herein. Additionally, the outer shell 300′ of FIGS. 4C through 4E is similar to the outer shell 300 of FIGS. 1 through 3, with the exception of the features discussed herein.

As shown in FIGS. 4C through 4E, the outer shell 200 is threadedly received within the adapter 500′ at the first end 510. In an example embodiment, the female threads 512 of the adapter 500′ are threadedly engaged with the male threads of the threaded end 210 of the outer shell 200. However, in other embodiments male threads can be provided on the adapter 500′ which threadedly engage female threads on the threaded end 210. In still other embodiments, any mating connection other than a threaded connection can be provided between the adapter 500′ and the outer shell 200.

Additionally, as shown in FIGS. 4C through 4E, the inner pin 300′ is received within the adapter 500′ at the second end 520. In an example embodiment, the inner pin 300′ is inserted within a cavity defined by an elongated portion 501 of the adapter 500′ until the abutment surface 322 of the inner pin 300′ abuts the lip 522 of the adapter 500′. Additionally, unlike the inner pin 300 of FIGS. 1 through 3, where the shaft 330 extends to the abutment surface 322, the inner pin 300′ of FIGS. 4C through 4E features an elongated portion 301 between the abutment surface 322 and the shaft 330. As shown in FIGS. 4C and 4D, the elongated portion 301 has an outer diameter 303 that is larger than the outer diameter 332 of the shaft 330 but smaller than an outer diameter of the knobbled end 320. In an example embodiment, the elongated portion 301 is inserted within the elongated portion 501 of the adapter 500′ such that a difference between the outer diameter 303 of the elongated portion 301 and an inner diameter 503 of the elongated portion 501 of the adapter 500′ may be in a range from about 0.0001 mm to about 0.1 mm or about 0.001 mm to about 0.01 mm. FIG. 4D further depicts that in one embodiment, unlike the adapter 500 of FIGS. 4A and 4B, the adapter 500′ features a tapered portion 524 between the elongated portion 501 and the first end 510.

FIGS. 5A, 5B, and 5C cooperate to illustrate the function of the surgical apparatus 100 and a method according to various embodiments. It should be noted that while FIGS. 5A through 5C depict and discuss the surgical apparatus 100 being used in conjunction with the sacroiliac joint 702, the surgical apparatus 100 can be used with any anatomical area or joint, using similar techniques and principles as discussed herein with respect to its use with the sacroiliac joint.

FIG. 5A is a schematic diagram of the surgical apparatus 100, in a first configuration 801, inserted into a sacroiliac joint 702. As shown, in this example embodiment of FIGS. 5A through 5C, the sacroiliac joint 702 is located on a human body 700 between a sacrum 704 and either a left or a right ilium 706. The surgical apparatus 100 may be inserted in the first configuration 801 directly into the sacroiliac joint 702. FIG. 5B is a schematic diagram of the surgical apparatus 100, in a second configuration 802, inserted into a sacroiliac joint 702 with an adapter 500 connected thereto to facilitate an injection of a substance 602 from a syringe 600 according to various embodiments. In other embodiments, the adapter 500′ of FIGS. 4C through 4E could be utilized in a similar manner as the adapter 500 of FIG. 5B to facilitate injection of the substance 602 from the syringe 600 into the sacroiliac joint 702. FIG. 5C is a schematic diagram of the surgical apparatus 100, in a second configuration 802, inserted into a sacroiliac joint 702 with an additional surgical instrument or treatment 900 sliding over the device 100, specifically over the outer shell 200, as may traditionally be accomplished with a Steinmann pin or an intermedullary pin.

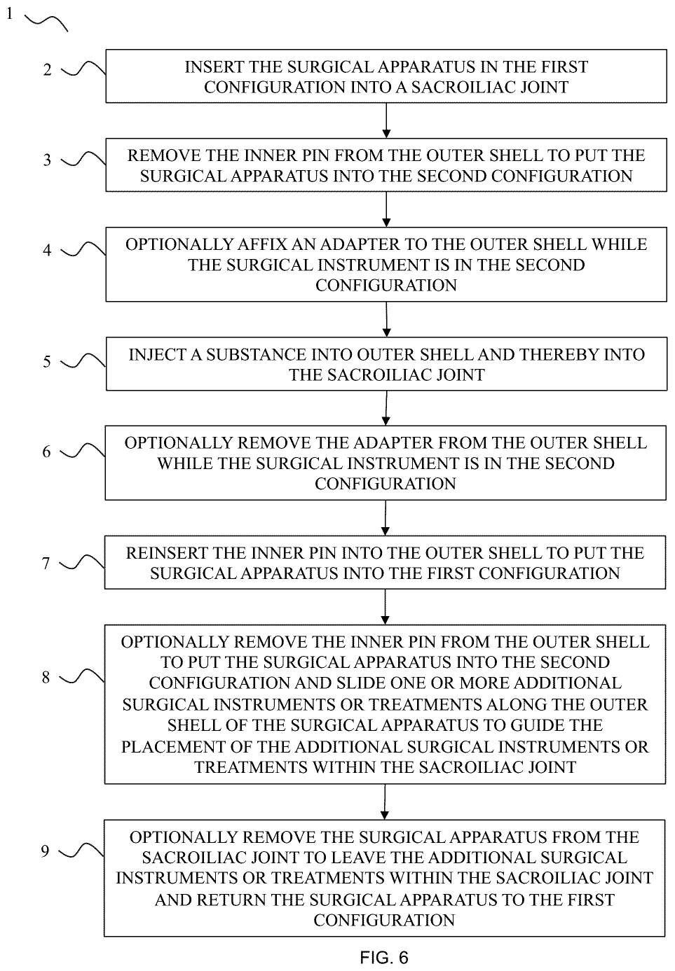

FIG. 6 is a block diagram illustrating a method 1, according to various embodiments, for injecting a substance 602 into an anatomical area (e.g. sacroiliac joint 702) via a surgical apparatus 100. The method 1 includes one or more of the following steps. Although steps are depicted in FIG. 6 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

The method 1 may comprise a step 2 of inserting the surgical apparatus 100 in the first configuration 801 into an anatomical area or joint (e.g. the sacroiliac joint 702). For purposes of the method 1, the remainder of the steps will be discussed with how the method 1 is performed on the sacroiliac joint 702. However, the method 1 can also be employed in any other anatomical area or joint using similar techniques and principles as discussed with respect to the sacroiliac joint 702.

In some embodiments, in step 2 the surgical apparatus 100 of FIG. 1 (e.g. the inner pin 300 received within the outer shell 200) in the first configuration 801 is inserted into the sacroiliac joint 702. In other embodiments, in step 2 the surgical apparatus 100′ of FIGS. 4C through 4E (e.g. the inner pin 300′ and outer shell 200 secured to opposite sides 510, 520 of the adapter 500′) in the first configuration 801′ is inserted into the sacroiliac joint 702.

For purposes of step 2, the surgical apparatuses 100, 100′ of FIG. 1 and FIG. 4C are similar, with the exception that in FIG. 4C the adapter 500′ is already in place.

The method 1 may comprise a step 3 of removing the inner pin 300 from the outer shell 200 to put the surgical apparatus 100 into the second configuration 802. In one embodiment, once the surgical apparatus 100′ of FIG. 4C is inserted into the joint in step 2, the inner pin 300 would be removed if the surgeon wants to inject a medication. Alternatively, if the surgical apparatus 100 of FIG. 1 is inserted into the joint in step 2, the surgeon removes the inner pin 300 and then connects the adapter 500 to the outer shell 200 (step 4 below) before injecting a medication.

In another embodiment, if the surgeon knows they don't want to use the surgical apparatus to inject a medication, in step 2 the surgeon would likely insert the surgical apparatus 100 of FIG. 1 into the sacroiliac joint 702. Then the surgeon may or may not remove the inner pin 300 in step 3 (e.g. depending on which surgical instrument they are going to pass over the inner pin 300) and use the surgical apparatus 100 like a traditional Steinman pin. If the surgeon later changed their mind and decided they want to inject a medication at any point, they would have to remove the inner pin 300 in step 3 and then attach the adapter 500 (step 4) before injecting medication.

In yet another embodiment, if the surgeon knows they are going to use the surgical apparatus to inject a medication, they would likely configure the surgical apparatus 100′ as shown in FIG. 4C. Thus, the surgeon would insert the surgical apparatus 100′ of FIG. 4C into a joint in step 2 and then in step 3 remove the inner pin 300 and could then inject a medication by inserting a syringe along the interior channel 240 of the outer shell 200 into the sacroiliac joint 702.

A lot of how the surgical apparatus is used will be dependent on various factors including but not limited to the nature of the procedure, which surgical instruments are being used in the case, as well as surgeon preference. Thus, the surgical apparatus advantageously provides versatility so to accommodate each of these factors of use.

In some embodiments, where step 2 involves insertion of the surgical apparatus 100 of FIG. 1, step 3 involves disengaging the abutment surface 322 of the inner pin 300 from the threaded end 210 of the outer shell 200. In other embodiments, where step 2 involves insertion of the surgical apparatus 100′ of FIGS. 4C through 4E, step 3 involves disengaging the abutment surface 322 of the inner pin 300 from the lip 522 of the second end 520 of the adapter 500′.

The method 1 may comprise an optional step 4 of affixing an adapter 500 to the outer shell 200 while the surgical instrument 100 is in the second configuration 802. In some embodiments, where step 3 involved the disengagement of the abutment surface 322 of the inner pin 300 from the threaded end 210 of the outer shell 200, step 4 involves threading the female threads 512 at the first end 510 of the adapter 500 over the male threads of the threaded end 210 of the outer shell 200. In other embodiments, where step 3 involved disengagement of the abutment surface 322 of the inner pin 300 from the lip 522 of the second end 520 of the adapter 500′, step 4 is omitted since the adapter 500′ is already affixed to the outer shell 200.

The configuration that includes optional step 4 allows the operator to use the adapter 500 to attach a syringe 600 (FIG. 5B) to inject a substance 602 (e.g. medication). There are a variety of situations where a surgeon may want to utilize the configuration to inject a medication or other therapeutic substance. In one example embodiment, they may want to inject a local anesthetic to help anesthetize the surgical field. In another example embodiment, they may want to inject an antibiotic for infection treatment or prophylaxis. In yet another example embodiment, they may want to inject a dye to ensure that a blood vessel or nerve or other structure hasn't been compromised. In yet another example embodiment, they may want to simply ensure that the tool is entering the correct space. In yet another example embodiment, they may want to inject a demineralized bone matrix to aid in bony fusion, or a biologic agent as a therapy. In yet other example embodiments, chemotherapeutic agents and neurolytic agents could be injected.

If the operator does not desire to inject a medication, they can decide not to exercise optional step 4 and instead they use it in the other configuration, which allows the surgical instrument to function as a traditional Steinmann pin or guide wire type device.

The method 1 may comprise a step 5 of injecting the substance 602 into the outer shell 200 and thereby into the sacroiliac joint 702. In some embodiments, in step 5 the syringe 600 connects to the adapter 500 (e.g. also called a leur lock in the medical field). The adapter 500 attaches to the outer shell 200. Thus, in these embodiments the medication or substance 602 contained within the syringe 600 would flow from the syringe 600, through the adapter 500 and outer shell 200 and would exit from the tapered end 222 to reach its final target (e.g., a joint or other anatomic space). In some embodiments, after affixing the adapter 500 to the outer shell 200 in step 4, in step 5 the syringe 600 is inserted into the second end 520 of the adapter 500 and through the interior channel 240 of the outer shell 200 to beyond the tapered end 220 and into the sacroiliac joint 702. In other embodiments, in step 5 the syringe 600 is inserted through the second end 520 of the adapter 500′ and through the interior channel 240 of the outer shell 200 beyond the tapered end 220 and into the sacroiliac joint 702.

The inventor of the present invention recognized that the injecting step 5 is an improvement over the prior art because with this surgical apparatus a therapeutic substance 602 (e.g. medication) can be injected. The surgical apparatus can also function as a guide wire or traditional Steinmann pin. Thus, it advantageously has dual operability, which a conventional Steinmann pin does not, and thus ultimately gives the operator a versatile tool when performing surgery.

The inventor of the present invention further recognized that the surgical apparatus herein is advantageous over conventional surgical devices since it replaces multiple surgical devices (e.g. guide wire and injection device) and thus advantageously involves introducing less foreign bodies into the subject during surgery, thereby reducing the risk of infection.

Additionally, in some embodiments, the surgical apparatus is constructed with a rigidity that is comparable with that of the traditional Steinmann pin. Unlike the traditional Steinmann pin with a solid interior, when used in the second configuration 802 the surgical apparatus 100 features the outer shell 200 with the interior channel 240. The inventors constructed the outer shell 200 with the interior channel 240 so to have comparable rigidity as the traditional Steinmann pin and thus capable of withstanding routine axial and rotational forces imparted thereon during surgical procedures (e.g. being struck with a hammer, undergoing twists as it is inserted into various joints, etc.) without significantly bending or breaking.

The method 1 may comprise an optional step 6 of removing the adapter 500 from the outer shell 200 while the surgical instrument 100 is in the second configuration 802. As previously discussed herein, the surgical apparatus can advantageously act as a traditional Steinmann pin or a needle for injection or both. In some embodiments, optional step 6 may be exercised in certain scenarios, such as after using the surgical apparatus as a needle for injection (step 5), the operator may want to utilize the surgical apparatus as a guide to glide another surgical instrument over the surgical apparatus. This optional step 6 is the first step towards this use transition.

The method 1 may comprise a step 7 of reinserting the inner pin 300 into the outer shell 200 to put the surgical apparatus 100 into the first configuration 801. In some embodiments, step 7 is performed in a similar manner as step 2. In some embodiments, the reinserting step 7 is exercised depending on various factors. In one example embodiment, the reinserting step 7 is performed if fluid is coming out of the outer shell 200. For example, if the surgical apparatus 100 in the first configuration 801 is inserted into a joint, after which the inner pin 300 is removed and joint fluid starts to flow back out of the outer shell 200 through the interior channel 240, this fluid would need to be stopped. Hence, in this example embodiment step 7 would be performed to reinsert the inner pin 300 back into the outer shell 200 to stop this fluid flow. This step would also be performed if other material, such as blood or medication that was recently injected starts to flow back out of the outer shell 200 after removing the inner pin 300.

In another embodiment, step 7 may be performed in order to enhance the structural stability to the outer shell 200 by inserting the inner pin 300 within the interior channel 240. In one example embodiment, if the operator decides to slide a surgical apparatus 900 over the tip of the surgical apparatus 100, the surgical apparatus 900 may be bulky and thus it would be prudent to enhance the structural stability of the surgical apparatus 100 prior to this step. Additionally, yet another reason to enhance the structural stability of the surgical apparatus 100 by inserting the inner pin 300 within the outer shell 200 is to withstand forces applied to the surgical apparatus 100, such as forces from a hammer used to knock the surgical apparatus 100 into a joint. Furthermore, different joints have various shapes and sizes which may tend to impose torque on and in the absence of this enhanced structural stability, may tend to bend the surgical apparatus 100 as it is forced therein. Hence, this is an additional reason to enhance the structural stability of the surgical apparatus by performing step 7.

The method 1 may comprise an optional step 8 of removing the inner pin 300 from the outer shell 200. After removing the inner pin 300, step 8 involves sliding one or more additional surgical instruments or treatments 900 along the outer shell 200 of the surgical apparatus 100 preferably in the second configuration 802 to guide the placement of the additional surgical instruments or treatments 900 within the sacroiliac joint 702. In some embodiments, depending on which surgical instrument 900 the operator is sliding over the surgical apparatus (e.g. its size), the operator will decide whether the inner pin 300 needs to be removed from the outer shell 200 in step 8. Since the inner pin 300 has a knobbed end 320 with a lip on it, this lip may cause certain surgical instruments to get caught on it. Thus, for those particular surgical instruments, it would be advantageous to remove the inner pin 300 in step 8 prior to sliding the surgical instrument along the surgical apparatus 100.

In some embodiments, the surgical instrument 900 is a hypothetical separate surgical instrument. In one embodiment, the surgical instrument 900 resembles a trocar (e.g. a metal tube used in surgery that acts as a channel to facilitate passing surgical instruments through a small space). Such surgical instruments are typically used in conjunction with a tiny incision where the surgeon passes a trocar through that tiny incision. When using the surgical apparatus of the outer shell 200 in the second configuration 802 (e.g. as a Steinman pin), it acts as a guide to the trocar so the trocar can be inserted and end up in the correct position. Without the outer shell 200 acting as the Steinman pin, the surgical instrument 900 (e.g. trocar) could veer off course and end up in the wrong position.

On the other hand, with the addition of the syringe 600, the substance 602 (e.g. medication) can be injected into the joint (e.g. sacroiliac joint 702), such as a local anesthetic, dye, biologic substance, methyl methacrylate, etc. Injecting the substance 602 allows the surgical apparatus 100 to serve an additional and completely different purpose than the traditional Steinmann pin. In these embodiments, the inventors design of the surgical apparatus 100 advantageously is a marriage of a needle with a Steinman pin and thus can function as either one of these ubiquitous and important medical tools.

In one example embodiment, when doing a surgery such as a sacroiliac joint fusion, the entire surgery can be done under just a local anesthetic because the surgeon would be able to inject a local anesthetic through the surgical apparatus 100 (acting as a Steinman pin) and anesthetize the surgical field and then also pass instruments (e.g. surgical instrument 900) over the surgical apparatus 100 to finish the surgery. In these example embodiments, there is no need for a general anesthetic and this would be safer and cost much less compared to how conventional surgery is currently undertaken. Additionally, the surgical apparatus could also be used for other surgeries.

The method 1 may comprise an optional step 9 of removing the surgical apparatus 100 from the sacroiliac joint 702 to leave the additional surgical instruments or treatments 900 within the sacroiliac joint 702 and then returning the surgical apparatus 100 to the first configuration 801.

As discussed with respect to other embodiments, the adapter 500, 500′ comprises a first end 510 and a second end 520, the first end 510 being configured to threadedly engage the threaded end 210 of the outer shell 200, the second end 520 being configured to engage a syringe 600. In these embodiments, the step 5 of injecting a substance 602 into the outer shell 200 may comprise injecting the substance 602 into the outer shell 200 via the adapter 500, 500′. The substance 602 that is injected flows all the way from the first end 510 of the adapter 500, through the interior channel 240 of the outer shell 200 and out of the 500/210 end, through channel 240, and out the tapered end 220 of the outer shell 200 and into the sacroiliac joint 702. The substance 602 is a therapeutic substance. For example, the therapeutic substance may be selected from an anesthetic (e.g. local or general), a corticosteroid, an antibiotic, and combinations thereof. The substance 602 may be a diagnostic substance. For example, the diagnostic substance may be a dye, a contrast agent, and combinations thereof. Additionally, the list of surgical instruments 900 that can be used is exhaustive and includes any and all surgical instruments appreciated by one skilled in the art.

FIG. 7 is a schematic diagram illustrating a cross-sectional view of a surface 10 having surface irregularities 11. This figure helps to define the term “surface irregularity” as used herein. As shown in FIG. 7, the surface 10 has a plurality of surface irregularities 11, including both protrusions 12 and indentations 13. The protrusions 12 may be bumps, irregularities, or intentionally formed surface features like threads or fins. The indentations 13 may scratches, or intentionally formed surface features like through holes or grooves. An average surface level 14 may be determined and may represent an average level about which the peaks and troughs of the various surface features are distributed. A non-conforming surface irregularity 15 may be a surface irregularity 11, such as a protrusion 12 or an indentation 13, that has a peak 16 at a height 18 or a trough 17 at a depth 19 that exceeds a predetermined threshold. A surface irregularity may, therefore, be measured relative to the average surface level and may be deemed to be conforming or non-conforming. For example, the phrase a surface having no surface irregularity of greater than about 0.001 mm would indicate that no surface irregularity may have a height or a depth relative to the average surface level of the surface greater than about 0.001 mm. The inventor recognized that the surfaces having no surface irregularities greater than the identified value is advantageous for various reasons, depending which surface is in question. For example, the surface of the inner pin 300 should be relatively smooth so that it can be inserted easily into the interior channel 240 of the outer shell 200. Thus, the interior channel 240 inner surface should also be smooth. On the other hand, the inner surface of the interior channel 240 may have some irregularities that will give it some small but necessary amount of friction between the inner pin 300 and outer shell 200. Some amount of friction may be beneficial so that the inner 300 will stay somewhat inside of the interior channel 240 without falling out if held upright. In this example embodiment, the fit between the inner pin 300 and outer shell 200 should be a tight fit but also be easily removed by hand but not too easily such that it would simply disassemble if it became too slippery. The inventor recognized that some surface irregularity may be advantageous on the smooth segment 230 of the outer shell 200 so that it can glide into joints but not be too slippery (i.e., having tactile manual feedback and being able to feel tissues or anatomical layers could be of benefit).

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A surgical apparatus for insertion into an anatomical area, the apparatus comprising:
an outer shell comprising:
a threaded end,
a tapered end having a first tapered surface, and
a smooth segment extending therebetween,
wherein the outer shell defines an interior channel,
wherein the interior channel extends through the threaded end, the smooth segment, and the tapered end along a longitudinal axis of the outer shell; and
an inner pin comprising:
a pointed end having a second tapered surface,
a knobbed end, and
a shaft;
wherein the inner pin and the outer shell cooperatively engage to provide a first configuration and a second configuration;
wherein in the first configuration:
the inner pin and the outer shell are aligned along the longitudinal axis,
the inner pin is disposed within the outer shell,
the knobbed end of the inner pin abuts the threaded end of the outer shell,
the pointed end of the inner pin extends beyond the tapered end of the outer shell,
the first tapered surface and the second tapered surface align at a common angle relative to the longitudinal axis; and
wherein in the second configuration the inner pin is not disposed within the outer shell.

2. The surgical apparatus according to claim 1, wherein the interior channel is cylindrical, wherein the shaft is cylindrical, and wherein an inner diameter of the interior channel is within about 0.001 mm of an outer diameter of the cylindrical shaft.

3. The surgical apparatus according to claim 1, wherein in the first configuration, the first tapered surface and the second tapered surface align at the common angle relative to the longitudinal axis to form a third tapered surface, and wherein the third tapered surface has no surface irregularity of greater than about 0.001 mm.

4. The surgical apparatus according to claim 1, wherein the smooth segment of the outer shell has no surface irregularity of greater than about 0.001 mm and wherein the smooth segment of the outer shell does not comprise any through holes.

5. The surgical apparatus according to claim 1, wherein the common angle is in a range of from greater than about 90 degrees to less than about 180 degrees.

6. The surgical apparatus according to claim 1, in the first configuration, an abutment surface of the knobbed end of the inner pin abuts the threaded end of the outer shell and prevents the inner pin from extending further into the outer shell.

7. The surgical apparatus according to claim 1, further comprising an adapter, the adapter comprising a first end and a second end, the first end being configured to threadedly engage the threaded end of the outer shell, the second end being configured to engage a syringe.

8. The surgical apparatus according to claim 1, wherein in the first configuration, the apparatus has a length along the longitudinal axis from a tip of the pointed end of the inner pin to a proximal surface of the knobbed end of the inner pin is in a range of from about 50 mm to about 300 mm.

9. The surgical apparatus according to claim 1, wherein the shaft of the outer shell is cylindrical and wherein an outer diameter of the outer shell is about 0.5 to about 15 mm.

10. The surgical apparatus according to claim 1, wherein the anatomical area is a sacroiliac joint.

11. A method for injecting a substance into an anatomical area via a surgical apparatus, the apparatus comprising:
an outer shell comprising:
a threaded end,
a tapered end having a first tapered surface, and
a smooth segment extending therebetween,
wherein the outer shell defines an interior channel,
wherein the interior channel extends through the threaded end, the smooth segment, and the tapered end along a longitudinal axis of the outer shell; and
an inner pin comprising:
a pointed end having a second tapered surface,
a knobbed end, and
a shaft;
wherein the inner pin and the outer shell cooperatively engage to provide a first configuration and a second configuration;
wherein in the first configuration:
the inner pin and the outer shell are aligned along the longitudinal axis,
the inner pin is disposed within the outer shell,
the knobbed end of the inner pin abuts the threaded end of the outer shell,
the pointed end of the inner pin extends beyond the tapered end of the outer shell,
the first tapered surface and the second tapered surface align at a common angle relative to the longitudinal axis; and
wherein in the second configuration the inner pin is not disposed within the outer shell,
the method comprising:
inserting the surgical apparatus in the first configuration into the anatomical area, removing the inner pin from the outer shell to put the surgical apparatus into the second configuration, and
injecting the substance into outer shell and thereby into the anatomical area.

12. The method according to claim 11, further comprising reinserting the inner pin into the outer shell to put the surgical apparatus into the first configuration.

13. The method according to claim 11, further comprising sliding one or more additional surgical instruments or treatments along the outer shell of the surgical apparatus in the first configuration to guide the placement of the additional surgical instruments or treatments within the anatomical area.

14. The method according to claim 13, further comprising removing the surgical apparatus from the anatomical area to leave the additional surgical instruments or treatments within the anatomical area.

15. The method according to claim 11, further comprising affixing an adapter to the outer shell while the surgical instrument is in the second configuration.

16. The method according to claim 15, wherein the adapter comprises a first end and a second end, the first end being configured to threadedly engage the threaded end of the outer shell, the second end being configured to engage a syringe, the method further comprising injecting the substance into the outer shell via the adapter.

17. The method according to claim 11, wherein the substance a therapeutic substance.

18. The method according to claim 17,
wherein the therapeutic substance is selected from the group consisting of an anesthetic, a corticosteroid, an antibiotic, and combinations thereof; or
wherein the diagnostic substance is selected from the group consisting of a dye, a contrast agent, and combinations thereof.

19. The method according to claim 11, wherein the substance is diagnostic substance.

20. The method according to claim 11, wherein the anatomical area is a sacroiliac joint.
